(19) United States
(12) Patent Application Publication
(10) Pub. No.: US 2010/0215635 A1
(43) Pub. Date: Aug. 26, 2010

(54) SMALL MOLECULES TO INDUCE WEIGHT LOSS OR TO REDUCE WEIGHT GAIN

(76) Inventor: Zoltan Kiss, Austin, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/391,006

(22) Filed: Feb. 23, 2009

(51) Int. Cl. A61K 38/46; A61K 31/382; A61P 3/04

(52) U.S. Cl. 424/94.6; 514/437

(57) ABSTRACT

Embodiments of the present invention include the use of heterocyclic trialkyl ammonium-containing compounds alone or in combination with alkaline phosphatase or other weight loss-inducing drugs or methods to reduce or reverse excess weight gain and obesity as well as reduce the risk of development and help treatment of diseases and unhealthy conditions related to overweight and obesity.

### TECHNICAL FIELD

The invention generally relates to the use of a family of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl] trimethylammonium chloride, or CCompound1, N,N,-diethyl-N-methyl-2-[9-oxo-9H-thioxanthen-2-yl)methoxy]ethanaminium iodide, or CCompound3, and N,N,N-trimethyl-3-(9H-thioxanthen-9-ylidene)-propane-1-aminium iodide, or CCompound19, to reduce or reverse excess weight gain and obesity as well as reduce the risk of development and help treatment of diseases and unhealthy conditions related to overweight and obesity. The heterocyclic compounds may be used alone or in combination with alkaline phosphatase or other weight loss-inducing drugs and methods.

### BACKGROUND

Results from the 2003-2004 National Health and Nutrition Examination Survey estimates that about 65% of US adults are either overweight or obese. Another recent estimate indicates that about 18.9% of the total US population and 31% of US adults are obese. The prevalence of overweight among children in the US has been more than doubled between the 1960s and 1988-1994 and is expected to further increase beyond 2010. In 2007, about 1.7 billion people worldwide were overweight or obese. An overweight person has a body mass index (BMI) between 25.0-29.9 and an obese subject has a BMI greater than or equal to 30.0. BMI is expressed as weight in kilograms (or pounds) divided by their height in meters (or inches) squared [Sonnenberg, G. E., Matfin, G. and Reinhardt, R. R. (2007) Drug treatments for obesity: where are we heading and how do we get there? Br. J. Diabetes Vasc. Dis. 7, 111-118; Ogden, C. L., Flegal, K. M., Carroll, M. D. and Johnson, C. L. (2002) Prevalence and trends in overweight among US children and adolescents. 1999-2000. JAMA 288, 1728-1732].

Overeating and reduced physical activity are considered to be the two major reasons of obesity, although several additional factors may also contribute [Jeffery, R. W. and Harnack, L. J. (2007) Evidence implicating eating as a primary driver for the obesity epidemic. Diabetes 56, 2673-2676; Hamilton, M. T., Hamilton, D. G. and Zderic, T. W. (2007) Role of low energy expenditure and sitting in obesity metabolic syndrome type 2 diabetes and cardiovascular disease. Diabetes 56, 2655-2667]. Unfortunately, some of the frequently used anti-diabetic agents also cause weight gain for which the best example is insulin. Other anti-diabetic agents causing weight gain include the thiazolidinedione class of compounds (such as Rosiglitazone and Pioglitazone), the sulfonylurea class of drugs (Tolbutamide, Glyburide), and Repaglinide (a meglitinide class drug). Thus, in a sense, while these drugs are useful by promoting removal of excess glucose from the blood, they also add to the problem by increasing adiposity. Other drugs that may enhance weight gain include atypical antipsychotics (olanzapine, clozapine), antidepressants, mood stabilizers, anticonvulsants, steroid hormones, beta-blockers, oral contraceptives, antihistamines, HIV antiretroviral drugs and protease inhibitors [Keith, S. W., Redden, D. T., Katzmarzyk, P. T., Boggiano, M. M., Hanlon, E. C., Bence, R. M., Ruden, D., Pietrobelli, A., Barger, J. L., Fontaine, K. R., Wang, C., Aronne, L. J., Wright, S. M., Baskin, M., Dhurandhar, N. V., Lijoi, M. C., Grilo, C. M., DeLuca, M., Westfall, A. O. and Allison, D. B. (2006) Putative contributors to the secular increase in obesity: exploring the roads less traveled. International. J. Obesity. 30, 1585-1594].

Obesity is a risk factor for many diseases and unhealthy conditions including high blood pressure, insulin resistance or impaired glucose tolerance, hyperinsulinemia, metabolic syndrome, type 2 diabetes that with time may transition into type 1 diabetes, stroke, heart attack, heart failure, atherosclerosis, inflammation, coagulation, fibrinolysis, certain types of cancer, gallstones, gout and gouty arthritis, osteoarthritis, sleep apnea, pickwickian syndrome, periodontal disease, abdominal hernias, varicose veins, renal failure, dementia, and liver malfunction [Must, A., Spadano, J., Coakley, E. H., Field, A. E., Colditz, G. and Dietz, W. H. (1999) The disease burden associated with overweight and obesity. JAMA 282, 1523-1529; Horwich, T. B., Fonarow, G. C., Hamilton, M. A., MacLellan, W. R., Woo, M. A. and Tillisch, J. H. (2001) The relationship between obesity and mortality in patients with heart failure. J. Am. Coll. Cardiol. 38, 789-795; Davy, K. P. and Hall, J. E. (2004) Obesity and hypertension: two epidemics or one? Am. J. Physiol. Regul. Integr. Comp. Physiol. 286, R803-R813; Erdmann, J., Kallabis, B., Oppel, U., Sypchenko, O., Wagenpfeil, S, and Schusdziarra, V. (2008) Development of hyperinsulinemia and insulin resistance during the early stage of weight gain. Am. J., Physiol. Endocrinol. Metab. 294, E568-E575; Goran, M. L., Ball, G. D. and Cruz, M. L. (2003) Obesity and risk of type 2 diabetes and cardiovascular disease and children and adolescents. J. Clin. Endocrinol. Metab. 88, 1417-1427; Lau, D. C. W., Dhillon, B., Yan, H., Szmitko, P. E. and Verma, S. (2005) Adipokines: molecular links between obesity and atherosclerosis. Am. J. Physiol. Heart Circ. Physiol. 288, H2031-H2041]. Obesity is also an independent predictor of mortality following severe blunt trauma [Neville, A. L., Brown, C. V. R., Weng, J., Demetriades, D. and Velmahos, G. C. (2004) Obesity is an independent risk factor of mortality in severely injured blunt trauma patient. Arch. Surg. 139, 983-987]. One of the most devastating consequences of obesity is diabetes; about 60% of all type 2 diabetes cases is due to obesity [Runge, C. F. (2007) Economic consequences of the obese. Diabetes 56, 2668-2672]. Because of the risks obesity entails, someone who is 40% overweight is twice as likely to die prematurely compared to an average-weight person.

The private and social costs of obesity are large. According to a recent estimate, health care costs for overweight and obese individuals are 37% higher than for people of normal weight, adding an extra $ 732 to the health care bill of each and every American. In the US, obesity and related conditions are estimated to result in $62.7 billion in doctor's visits and $ 39.3 billion in lost workdays each year [Runge, C. F. (2007) Economic consequences of the obese. Diabetes 56, 2668-2672].

One of the current obesity treatment options includes reduced caloric intake and increased physical activity. These approaches however fail in 90% of cases. Studies show that diets and exercise programs initially result on average in about 10% weight loss, but obese subjects tend to regain two-thirds of weight within one year and most of the weight within five years. Another method to help lose weight is bariatric surgery that could lead to 21-38% loss of baseline weight. However, this procedure is accompanied with surgical-related risks and requires a life time adherence to extensive dietary, exercise, and medical guidelines. Phentermine is an appetite suppressant that is recommended for short-term use because of its possible side effects. In addition, a limited number of anti-obesity drugs are available for longer-term use. These include Orlistat that inhibits intestinal lipase enzymes [Muls, E., Kolanowski, J., Scheen, A. and Van Gaal, L. (2001) The effects of Orlistat on weight and on serum lipids in obese patients with hypercholesterolemia: a randomized double-blind placebo-controlled multicentre study. International J. Obesity 25, 1713-1721; Richelsen, B., Tonstad, S., Rossner, S., Toubro, S., Niskanen, L., Madsbad, S., Mustajoki, P. and Rissanen, A. (2007) Effect of orlistat on weight regain and cardiovascular risk factors following a very-low-energy diet in abdominally obese patients. Diabetes Care 30, 27-32], and Sibutramine that blocks re-uptake of monoamine neurotransmitters in the brain [Vettor, R., Serra, R., Fabris, R., Pagano, C. and Federspil, G. (2005) Effect of Sibutramine on weight management and metabolic control in type 2 diabetes. Diabetes Care 28, 942-949; Wirth, A. and Krause, J. (2001) Long-term weight loss with sibutramine: A randomized controlled trial. JAMA 286, 1331-1339]. Rimonabant (Acomplia) has been widely used in Europe during the last few years but it has not received the U.S. Food and Drug Administration's approval because of psychiatric effects. Rimonabant blocks binding of endogenous cannabinoid to neuronal CB1 receptors. While the future of Rimonabant is uncertain, other drug candidates are being developed that also interact with the CB1 receptor. Thus, Merck & Co. is developing Trabanant, a CB1 receptor agonist, and Pfizer is developing CP-945598, a CB1 receptor antagonist, to control weight gain. Each drug has side effects such as diarrhea, flatulence, bloating, abdominal pain, and sispepsia (Orlistat); dry mouth, constipation and insomnia (Sibutramine); nausea and mood disorders (Rimonabant).

### SUMMARY OF THE INVENTION

The present invention relates to the use of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl] trimethylammonium chloride, or CCompound1, N,N,-diethyl-N-methyl-2-[9-oxo-9H-thioxanthen-2-yl)methoxy]ethanaminium iodide, or CCompound3, and N,N,N-trimethyl-3-(9H-thioxanthen-9-ylidene)-propane-1-aminium iodide, or CCompound19 for preventing an increase in, or reducing, the body weight a subject that is overweight (BMI value between 25.0-29.9) or obese (BMI value greater than or equal to 30.0) as well as reducing risks causally related to being overweight or obese.

Several CC compounds significantly decreased body weight in a widely used experimental model of obesity using ob/ob mice. Ob/ob mice are genetically obese as a result of mutations in the leptin gene [Drel, V. R., Mashtalir, N., Ilnytska, O., Shin, J., Li, F., Lyzogubov, V. V. and Obrosova, I. G. (2006) The leptin-deficient (ob/ob) mouse: A new animal model of peripheral neuropathy of type 2 diabetes and obesity. Diabetes 55, 3335-3343]. Leptin plays a major regulatory role in nutrition, body weight, and physiology [Friedman, J. M. (2002) The function of leptin in nutrition, weight, and physiology. Nutritional Rev. 60, S1-S14]. In the absence of leptin, these mutant animals, just like leptin-deficient or leptin resistant humans, fail to sense that there are adequate fat stores. Thus ob/ob animals and humans with disregulated leptin function exist in a state of perceived starvation and in turn become overeaters and obese if food is available. In humans, leptin resistance, which has the same physiological consequence as leptin deficiency, plays a role in the majority of obesity cases [Chen, K., Li, F., Li, J., Cai, H., Strom, S., Bisello, A., Kelley, D. E., Friedman-Einat, M., Skibinski, G. A., McCrory, M. A., Szalai, A. J. and Zhao, A. Z. (2006) Induction of leptin resistance through direct interaction of C-reactive protein with leptin. Nature Med. 12, 425-432]. Therefore, it is generally accepted in the art that if a compound induces body weight changes in the ob/ob mouse model, then similar effects can be expected in overweight and obese human subjects.

In one embodiment, the CC compound is administered to an overweight subject with a BMI between 25.0-29.9. In another embodiment, the CC compound is administered to an obese subject with a BMI greater than or equal to 30.0.

In yet another embodiment the CC compound is administered to a normal-weight (BMI between 18.5-24.9), overweight, or obese subject to either prevent or reduce abnormal weight gain and thereby reduce the risk of excess weight-associated diseases and unhealthy conditions.

A suitable CC compound may be administered alone or along with an oral or injectable anti-obesity agent such as an alkaline phosphatase.

In a certain embodiment, the invention provides a treatment regimen for the treatment of an overweight or obese mammal comprising periodically administering a therapeutically effective amount of a suitable CC compound alone or together with another anti-obesity treatment.

In another embodiment, the treatment regimen for the treatment of a mammal with a CC compound is provided to improve or prevent complications resulting from excess weight and obesity.

In an additional embodiment, the invention provides for the use of a CC compound alone or together with another anti-obesity drug in the manufacture of a composition useful for the reduction of excess weight and obesity.

In some embodiments, the mammal is administered a therapeutically effective amount of a suitable CC compound such as CCompound1, CCompound3, or CCompound19. The term “therapeutically effective amount” is used in this application to mean a dose that is effective to measurably reduce body weight, or prevent weight gain compared to a baseline increase in weight during a period just prior to the treatment.

### DETAILED DESCRIPTION OF THE INVENTION

### Active Components

The compounds used in the application, collectively termed “CC compounds”, contain a heterocyclic moiety to which a quaternary ammonium-containing moiety is attached at one or more of the following positions: R₂, R₁₀, V or Y of the heterocyclic moiety represented by the formula:

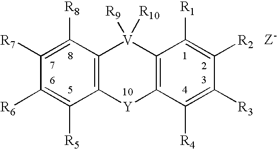

wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties; wherein R9 and R10 considered together are ═O or ═CH-L-N⁺(R11, R12, R13) or wherein R9 and R10 considered independently are —OH or -L-N⁺(R11, R12, R13);

wherein R2 is represented by the formula: —X or —X′-L-N⁺(R11, R12, R13)Z- or -L-N+(R11, R12, R13)Z⁻;

wherein V is —S—, —Se—, —C—, —O— or —N;

wherein Y is —S—, —Se—, —C—, —O— or —N;

wherein -L-N⁺(R11, R12, R13) can be linked to V or Y if V or Y is —N or can be linked to V and Y if V and Y are both —N;

wherein X is CH3 or Hydrogen;

wherein —X′ is -CH2-, -OCH2-, -CH2O-, -SCH2- or -CH2S-;

wherein L is a C1-C4 straight alkane, alkene, thiol, ether, alcohol, or amine;

wherein R11, R12 and R13 are independently Hydrogen, C1-C4 straight alkanes, alkenes, thiols,

amines, ethers or alcohols; and

wherein Z- is Cl⁻, Br⁻ or I⁻.

One embodiment of these compounds is [3-(3,4-Dimethyl-9-oxo-9H-thioxanthen-2-yloxy)-2-hydroxypropyl]trimethyl-ammonium chloride or CCompound1. CCompound1 was acquired commercially (Sigma-Aldrich) or synthesized by a method indicated for other CC compounds below. Two other embodiments of these compounds are N,N-diethyl-N-methyl-2-[(9-oxo-9H-thioxanthen-2-yl)methoxy]-ethanaminium iodide or CCompound3, and N,N,N-trimethyl-3-(9H-thioxanthen-9-ylidene)-propane-1-aminium iodide, or CCompound19. Exemplary methods of synthesizing representative CCompounds are described in U.S. patent application Ser. No. 11/458,502, filed Jul. 19, 2006, entitled “Compounds and compositions to control abnormal cell growth”; inventor: Zoltan Kiss, which is incorporated herein by reference in its entirety.

Table 1 shows a representative list of CC compounds that are predicted to share, at least in part, the effects of CCompound1, CCompound3 and CCompound19 on body weight.

TABLE 1
A Representative List of CCcompounds Used in the Invention.

| Trivial name | Chemical name | Structure |
|---|---|---|
| CCcompound 1 | [3-(3,4-Dimethyl-9-oxo- 9H-thioxanthen-2-yloxy)-2- hydroxypropyl]trimethyl- ammonium chloride | 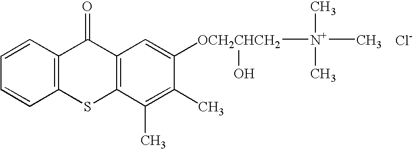 |
| CCcompound 2 | N,N,N-Trimethyl-2-[(9- oxo-9H-thioxanthen-2- yl)methoxy]- ethanaminium iodide | 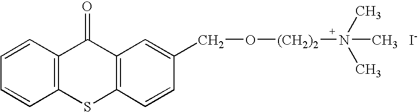 |
| CCcompound 3 | N,N-Diethyl-N-methyl-2- [9-oxo-9H-thioxanthen-2- yl)methoxy]- ethanaminium iodide | 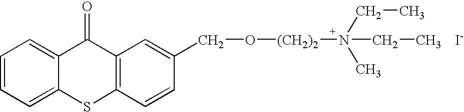 |
| CCcompound 4 | N,N,N-Triethyl-2-[(9-oxo- 9H-thioxanthen-2- yl)methoxy]- ethanaminium iodide | 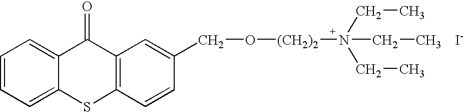 |
| CCcompound 5 | N-Ethyl-N,N-dimethyl-2- [(9-oxo-9H-thioxanthen- 2-yl)methoxy]- ethanaminium iodide | 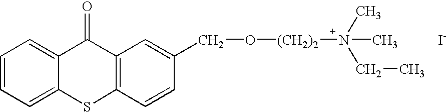 |
| CCcompound 6 | 2-{[2- (Diethylamino)ethoxy] methyl}-9H-thioxanthen- 9-one hydrochloride | 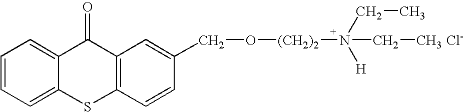 |
| CCcompound 7 | N,N,N-Trimethyl-2-[(9- oxo-9H-thioxanthen-2- yl)methoxy]-propan-1- aminium iodide | 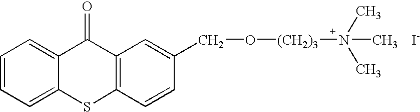 |
| CCcompound 8 | 2-{[2- (Dimethylamino)propoxy] methyl}-9H-thioxanthen- 9-one hydrochloride | 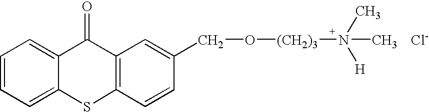 |
| CCcompound 9 | N,N,N-Triethyl-3-[(9-oxo- 9H-thioxanthen-2- yl)methoxy]-propane-1- aminium iodide | 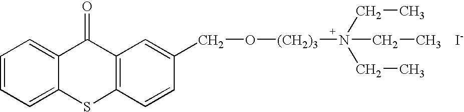 |
| CCcompound 10 | N,N-Diethyl-N-methyl-3- [(9-oxo-9H-thioxanthen- 2-yl)methoxy]-propane- 1-aminium iodide | 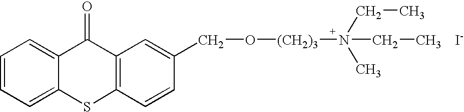 |
| CCcompound 11 | N,N-Dimethyl-N-ethyl-3- [(9-oxo-9H-thioxanthen- 2-yl)methoxy]-propane- 1-aminium iodide | 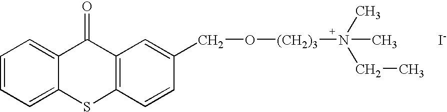 |
| CCcompound 12 | 2-{[3- (Diethylamino)propoxy] methyl}-9H-thioxanthen- 9-one hydrochloride | 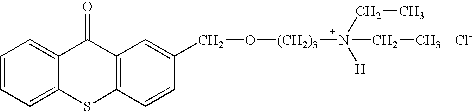 |
| CCcompound 13 | 2-Hydroxy-N,N-dimethyl- N-[(9-oxo-9H- thioxanthen-2-yl)methyl]- ethanaminium bromide | 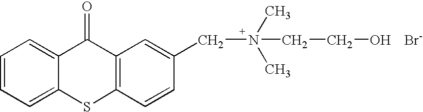 |
| CCcompound 14 | 2-Hydroxy-N,N-Diethyl- N-[9-oxo-9H- thioxanthen-2-yl)methyl]- ethanaminium bromide | 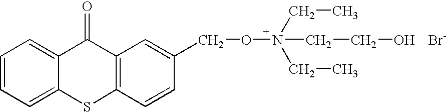 |
| CCcompound 15 | 3-Hydroxy-N,N-dimethyl- N-[(9-oxo-9H- thioxanthen-2- yl)methyl]propane-1- aminium bromide | 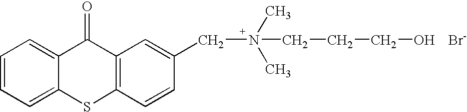 |
| CCcompound 16 | 3-Hydroxy-N,N-diethyl- N-[(9-oxo-9H- thioxanthen-2-yl)methyl]- propane-1-aminium bromide | 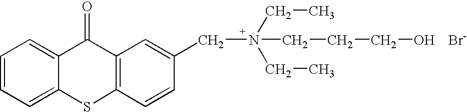 |
| CCcompound 17 | 3-(9-hydroxy-9H- thioxanthen-9-yl)-N,N,N- trimethyl-propane-1- aminium iodide | 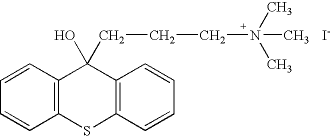 |
| CCcompound 18 | 3-(9-hydroxy-9H- selenoxanthen-9-yl)- N,N,N-trimethyl-propane- 1-aminium iodide | 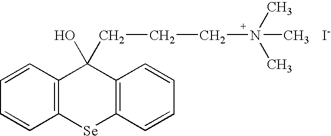 |
| CCcompound 19 | N,N,N-trimethyl-3-(9H- thioxanthen-9-ylidene)- propane-1-aminium iodide | 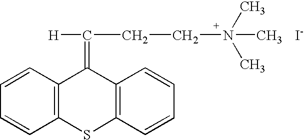 |
| CCcompound 20 | N,N,N-trimethyl-3-(9H- selenoxanthen-9-ylidene)- propane-1-aminium iodide | 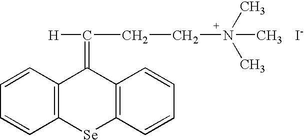 |
| CCcompound 21 | N,N,N-trimethyl-3-(2- methyl-9H-thioxanthen-9- ylidene)-propane-1- aminium iodide | 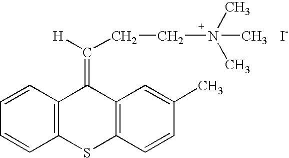 |
| CCcompound 22 | N,N-Dimethyl-N-ethyl-3- (2-methyl-9H- thioxanthen-9-ylidene)- propane-1-aminium iodide | 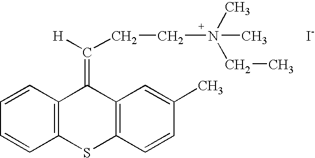 |
| CCcompound 23 | N,N-Diethyl-N-methyl-3- (2-methyl-9H- thioxanthen-9-ylidene)- propane-1-aminium iodide | 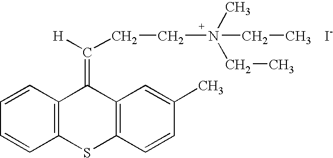 |
| CCcompound 24 | N,N-Dimethyl-N-allyl-3- (2-methyl-9H- thioxanthen-9-ylidene)- propane-1-aminium bromide | 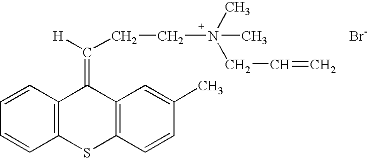 |
| CCcompound 25 | N,N,N-Triethyl-3-(2- methyl-9H-thioxanthen-9- ylidene)-propane-1- aminium iodide | 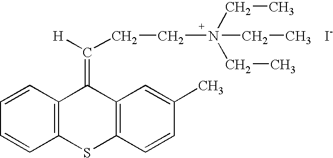 |
| CCcompound 26 | N,N-Diethyl-N-allyl-3-(2- methyl-9H-thioxanthen-9- ylidene)-propane-1- aminium bromide | 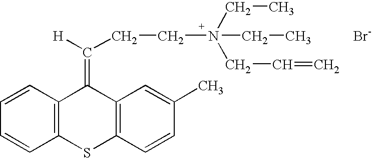 |

### Methods of Treatments

CC compounds used in this invention are well soluble in both water and dimethylsulfoxide. Accordingly, oral application is one of the major administration routes to deliver a CC compound. In one embodiment of the invention, the CC compound is in the form of a tablet, gel capsule, a liquid, or the like. In each case, the CC compound is mixed with one or more carriers chosen by one having ordinary skill in the art to best suit the goal of treatment. In addition to the active compounds, the tablet or gel capsule may contain any component that is presently used in the pharmaceutical field to ensure firmness, stability, solubility and appropriate taste. In some embodiments, additional components of the tablet or gel will be chemically inert; i.e., it will not participate in a chemical reaction with the CC compound or the additives.

CC compounds may also be applied via intravenous, intraarterial, intraportal, intradermal, intraperitoneal, subcutaneous, intra-tissue or intramuscular delivery routes. In some embodiments, the CC compound may be delivered via infusion over a period of time or by using an osmotic minipump inserted under the skin for controlled release. The injectable solution may be prepared by dissolving or dispersing a suitable preparation of the CC compound in water or water-based carrier such as 0.9% NaCl (physiological saline) or phosphate buffered saline. Alternatively, the CC compound may be dissolved first in dimethylsulfoxide and then diluted (100-400-fold dilution) in a physiologically compatible carrier using conventional methods. This latter method is particularly suitable if the preparation contains another drug, in addition to the CC compound, that first needs to be dissolved in dimethylsulfoxide. As an example only, a suitable composition for the practice in the method comprises a CC compound in a 0.9% physiological saline solution to yield a total CC compound concentration of 0.1-g/ml or 25.0-g/ml, or of 1.0-g/ml or 10.0-g/ml.

A suitable dosage for oral administration or for injection may be calculated in milligrams or grams of the active agent(s) per square meter of body surface area for the subject. In one embodiment, the therapeutically effective amount of CC compound is administered orally at a dose between 100-mg to 2,000-mg, or between 200-mg to 1,000-mg, per m² body surface of the mammal. In another embodiment, the CC compound is administered by an injection method at a dose of 50-mg to 1,000-mg, or between 100-mg to 500-mg, per m² body surface of the mammal.

The amount of the CC compound may vary depending on the method of application. For example, in case of intravenous application the required amount may approach the lower limit, while in case of subcutaneous application the required amount may be closer to the upper limit. Also, if oral application is repeated several times a day, the dose may be lowered compared to the once a daily application.

Application of the CC compound orally or by one of the above injection or infusion application methods may be repeated as many times as needed to achieve a satisfactory reduction in blood glucose level. However, for practical reasons, oral administration can be made more frequent than injection applications.

In one embodiment, the therapeutically effective amount of CC compound may be administered once daily. In another embodiment, the dose is administered twice or three times daily. In still another embodiment, administration of the CC compound is performed three-times a week. In yet another embodiment the CC compound may be administered intermittently; as an example, CC compound may be administered once daily for two weeks followed by a two to 4 weeks rest period that is followed by repeating the cycle as many times as needed.

The CC compound may be used before or after bariatric surgery to enhance the effect of this procedure. The CC compound may also be used in combination with any anti-obesity drug including, for example, Orlistat, Sibutramine, and a CB1 receptor-interacting drug. The CC compound is suitable to be used together with any weight loss-inducing reduced calorie diet.

In some embodiments, the CC compound may be administered in combination with a human alkaline phosphatase that has recently been shown to reduce weight gain in an obese animal model [Z. Kiss, U.S. Pat. No. 7,014,852, Alkaline phosphatase to induce weight loss or to reduce weight gain, which is herein incorporated by reference in its entirety]. There are four human alkaline phosphatase isoforms, including the intestinal, tissue non-specific (liver/kidney/bone), germ cell, and placental isoforms. These alkaline phosphatase isoforms show high degree of sequence identity each expressing phosphatase activity.

The CC compound may also be used to prevent or reduce weight gain induced by various weight-enhancing drugs in normal-weight, overweight, or obese subjects. Examples for such drugs include insulin, the thiazolidinedione class of compounds (such as Rosiglitazone and Pioglitazone), the sulfonylurea class of drugs (Tolbutamide, Glyburide), Repaglinide (a meglitinide class drug), atypical antipsychotics (olanzapine, clozapine), antidepressants, mood stabilizers, anticonvulsants, steroid hormones, beta-blockers, oral contraceptives, antihistamines, HIV antiretroviral drugs and protease inhibitors.

The CC compound may also be used together with any drug that is used to treat a disease or an unhealthy physiological condition where the treated subject is overweight or obese. Using the CC compound in these cases may reduce weight gain and thereby enhance the efficacy of the standard treatment and reduce the risk of mortality and morbidity. Examples for such diseases and unhealthy conditions include high blood pressure, insulin resistance or impaired glucose tolerance, hyperinsulinemia, metabolic syndrome, diabetes, stroke, heart attack, heart failure, atherosclerosis, inflammation, coagulation, fibrinolysis, certain types of cancer, gallstones, gout and gouty arthritis, osteoarthritis, sleep apnea, pickwickian syndrome, periodontal disease, abdominal hernias, varicose veins, renal failure, dementia, Alzheimer's disease, and liver malfunction.

In case of oral administration of the CC compound, the other treatments may be applied together with, or separately from, the CC compound. In case of injection application, the CC compound and the one or more drugs may be dissolved or suspended in the same physiologically compatible carrier substance, or they can be administered separately.

In one embodiment of the combination treatment when the CC compound is used together with a standard treatment, the CC compound is administered in the amounts described above and the one or more drugs are used at doses and frequencies as prescribed or recommended by a regulatory agency.

In one embodiment of the combination treatment in which the CC compound is used together with alkaline phosphatase, the CC compound may be used in the amounts indicated above and the alkaline phosphatase may be administered in the amounts of 100-mg to 2,000-mg, or 200-mg to 1,000-mg, per m² of body surface. In this method, the alkaline phosphatase is administered once, twice, or three-times a week by an injection method, while the heterocyclic compound is administered daily or intermittently orally or by an injection method.

### EXAMPLES

### Animals

Leptin-deficient ob/ob obese diabetic inbred, adult female mice, weighing 32-36 g at arrival, specified pathogen free (SPF) hygienic category from Charles River VRF₁, were used for these experiments. The ob/ob obese mouse is an extensively used animal model for the study of non-insulin-dependent-diabetes mellitus (NIDDM). The mutation was propagated in the C57BL/6J (BL/6) inbred strain. Homozygous obese (ob/ob) animals developed hyperglycemia, hyperinsulinemia and obesity. In these animals gluconeogenesis is enhanced despite their hyperinsulinemic state.

The animals were kept in macrolon cages at 22-24° C. and 50-60% humidity, with lighting regimen of 12/12 h light dark. The animals had free access tap water and were fed with a sterilized standard diet (Charles River VRF₁, autoclavable). The animals were cared for according to the “Guiding Principles for the Care and Use for Animals” based upon the Helsinki declaration. The ob/ob mice gained weight rapidly and develop a marked obesity by 5-6 weeks of age. Correspondingly, food intake was greatly increased. When arrived they were 5 weeks old (when experiment was performed with CC1) or 4.5 weeks old (when experiment was performed with CC3 and CC19); the treatments started 8 days later in the experiment performed with CC1 and 10 days later in the experiment performed with CC3 and CC19.

C57BL6 male mice (10-11 weeks old) were used as non-obese controls of ob/ob mice. These animals were kept under specified pathogen free (SPF) condition and they were fed a standard sterilized diet (Charles River) ad libitum.

### Treatments

Experimental groups selected from C57/BL or ob/ob mice were injected subcutaneously at regular intervals once daily for 14 days with CCompound1, CCompound3, or CCompound19 at the dose of 4.5-mg per kg. Other groups selected from C57/BL or ob/ob mice remained untreated during the entire length of the experiment. Body weight was measured every day or every second day for 14 days and then on days 21, 40, and 48. All untreated and treated groups included 5 animals except the CCompound1-treated group of ob/ob mice that included 9 animals. The data are expressed as the average ±standard error of mean.

### Example 1

### Effect of CCompound1 on the Body Weight of C57/BL Mice

This mouse strain served as the control to the ob/ob mice. On day 1, body weight was measured first followed by the treatment in the CCompound1-treated group. The treatments were repeated for 14 days. As shown in TABLE 2, untreated mice gained 2-g body weight between day 1 and 14, and 5.2-g between day 14 and 48. CCompound1 treated mice also gained 2-g body weight between day 1 and 14, and 4.2-g between day 14 and 48. This small difference between the two groups was not statistically significant. In a follow up experiment using the same treatment regimen, C57/BL mice treated with 6-mg per kg dose of CCompound1 gained 5.6-g weight gain over a 40 days period compared to 5.8-g weight gain in the control group over the same time period. Again, this small difference was not statistically significant. Overall the results indicate that in normal mice, CCompound1 in the dose range of 4.5-6-mg per kg has no effects on the body weight.

TABLE 2
Effects of CCcompound1 on body weight of C57/BL mice

Body weight (g)

| Day | No Treatment | CCcompound1 |
|---|---|---|
| 1 | 28.3 ± 0.6 | 28.0 ± 0.7 |
| 2 | 28.1 ± 1.0 | 27.7 ± 0.6 |
| 4 | 29.2 ± 0.9 | 28.2 ± 0.7 |
| 6 | 29.5 ± 0.8 | 28.7 ± 0.5 |
| 8 | 30.0 ± 0.7 | 28.8 ± 0.4 |
| 10 | 30.2 ± 0.6 | 29.6 ± 0.7 |
| 12 | 30.2 ± 0.4 | 30.3 ± 1.4 |
| 14 | 30.3 ± 0.7 | 30.0 ± 0.9 |
| 21 | 31.3 ± 1.1 | 30.7 ± 1.1 |
| 40 | 34.1 ± 0.8 | 33.9 ± 0.9 |
| 48 | 35.5 ± 0.9 | 34.2 ± 0.8 |

### Example 2

### Effects of CCompound1 on the Body Weight of ob/ob Mice

On day 1, body weight was measured first followed by the treatment in the CCompound1-treated group. The treatments were repeated for 14 days with no additional treatment performed during the rest of the observation period. As shown in TABLE 3, untreated mice gained 4.4-g body weight between day 1 and 14, and 11.6-g between day 14 and 48. In contrast, CCompound1 treated mice gained 2.6-g body weight between day 1 and 14, and also only 2.6-g between day 14 and 48. On day 14 and the following days the differences in body weight were statistically highly significant (P<0.001). It is particularly important that between day 14 and 48, weight gain in the treated group was minimal compared to the control group despite discontinuation of the treatment. This indicates that the effect of CCompound1 on weight control is long lasting. This feature allows intermittent use of CCompound1 and analogs for weight control.

In the experiment described in Table 3, the animals had full access to food at all the time. It is logical to assume that when CCompound1 is used together with calorie restriction, then CCompound1 can actually induce weight loss. A similar assumption can be made if the CC compound is used together with a weight loss-inducing drug including an alkaline phosphatase.

TABLE 3
Effects of CC1 treatment on the body weight of ob/ob mice

Body weight (g) *P < 0.001

| Day | No Treatment | CCcompound1 |
|---|---|---|
| 1 | 40.6 ± 0.8 | 40.4 ± 0.7 |
| 2 | 40.5 ± 0.9 | 40.4 ± 0.6 |
| 3 | 40.8 ± 0.8 | 40.6 ± 0.6 |
| 4 | 41.6 ± 1.0 | 40.8 ± 0.6 |
| 5 | 41.3 ± 0.8 | 40.7 ± 0.6 |
| 6 | 42.3 ± 0.8 | 41.0 ± 0.6 |
| 7 | 42.3 ± 0.7 | 41.5 ± 0.6 |
| 8 | 43.1 ± 0.8 | 41.9 ± 0.7 |
| 9 | 43.1 ± 0.6 | 42.1 ± 0.7 |
| 10 | 43.6 ± 0.6 | 42.4 ± 0.7 |
| 11 | 43.7 ± 0.7 | 42.6 ± 0.6 |
| 12 | 43.7 ± 0.6 | 42.7 ± 0.6 |
| 13 | 44.0 ± 0.6 | 42.9 ± 0.6 |
| 14 | 45.0 ± 0.7 | 43.0 ± 0.7* |
| 19 | 46.4 ± 0.6 | 44.3 ± 0.9* |
| 21 | 47.1 ± 0.6 | 44.5 ± 0.8* |
| 45 | 55.9 ± 0.5 | 45.6 ± 1.1* |
| 48 | 56.6 ± 0.5 | 45.6 ± 1.1* |

### Example 3

### Effects of CCompound3 and CCompound19 on the Body Weight of ob/ob Mice

On day 1, first body weight was measured, followed by the treatments with CCompound3 (5 mice) or CCompound19 (5 mice) as indicated in TABLE 4. The treatments were repeated for 14 days with no additional treatment performed during the rest of the observation period. One group of animals (5 mice) served as untreated controls. As shown in TABLE 4, untreated mice gained 4.8-g body weight between day 1 and 15, and 11.9-g between day 15 and 48. Mice treated with CCompound3 gained 3.3-g body weight between day 1 and 15, and 6.8-g between day 15 and 48. Mice treated with CCompound19 gained 2.6-g body weight between day 1 and 15, and 4.2-g between day 15 and 48. On day 40 and day 48 the differences between either of the treated group and the control group were statistically highly significant (P<0.001) with CC19 being somewhat more effective in retarding body weight than CCompound3. Again, both CCompound3 and particularly CCompound19 were effective reducing weight between day 15 and 48 despite discontinuation of the treatment after the last treatment on day 15. This data shows that the compounds listed in Table 1, as structural analogs of CCompound1, CCompound3 and CCompound19, have inhibitory effects on body weight gain in overweight and obese subjects.

TABLE 4
Effects of CCcompound3 and CCcompound19 on the body weight of
ob/ob mice.

| | Body weight (g) *P < 0.001 | | |
|---|---|---|---|
| Days | No Treatment | CCcompound3 | CCcompound19 |
| 1 | 37.4 ± 0.8 | 37.0 ± 0.6 | 38.1 ± 0.6 |
| 5 | 38.8 ± 1.1 | 37.5 ± 1.2 | 38.4 ± 0.7 |
| 10 | 40.3 ± 0.5 | 38.5 ± 0.9 | 39.7 ± 0.4 |
| 15 | 42.2 ± 1.0 | 40.3 ± 1.1 | 40.7 ± 0.5 |
| 20 | 44.3 ± 0.7 | 42.2 ± 0.5 | 42.1 ± 0.9 |
| 40 | 52.1 ± 1.5 | 45.9 ± 0.8* | 43.8 ± 1.3* |
| 48 | 54.1 ± 0.7 | 47.1 ± 1.0* | 44.9 ± 0.8* |

1. A method for treating an overweight or obese mammal to induce weight loss or reduce weight gain, the method comprising administering a heterocyclic compound in a therapeutically effective amount to induce weight loss, to reduce an expected weight gain, or to maintain a constant body weight for the mammal over time, wherein the heterocyclic compound is represented by the formula: 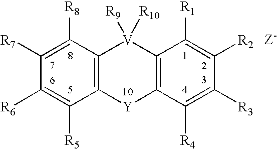
wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties;
wherein R9 and R10 considered together are ═O or ═CH-L-N⁺(R11, R12, R13) or wherein R9 and R10 considered independently are —OH or -L-N⁺(R11, R12, R13);
wherein R2 is represented by the formula: —X or —X′-L-N⁺(R11, R12, R13)Z- or -L-N⁺(R11, R12, R13)Z⁻;
wherein V is —S—, —Se—, —C—, —O— or —N;
wherein Y is —S—, —Se—, —C—, —O— or —N;
wherein -L-N⁺(R11, R12, R13) can be linked to V or Y if V or Y is —N or can be linked to V and Y if V and Y are both —N;
wherein X is CH₃ or Hydrogen;
wherein —X′ is -CH2-, -OCH2-, -CH2O-, -SCH2- or -CH2S-;
wherein L is a C1-C4 straight alkane, alkene, thiol, ether, alcohol, or amine;
wherein R11, R12 and R13 are independently Hydrogen, C1-C4 straight alkanes, alkenes, thiols, amines, ethers or alcohols; and
wherein Z- is Cl⁻, Br or I⁻.

2. The method of claim 1 wherein the mammal is a human subject.

3. The method of claim 1 wherein the mammal is expected to gain weight because of treatment with one or more weight-enhancing drugs exemplified by insulin, Rosiglitazone, Pioglitazone, Tolbutamide, Glyburide, Repaglinide, olanzapine, clozapine, an antidepressant, a mood stabilizer, an anticonsulvant, a steroid hormone, a beta-blocker, an oral contraceptive, an antihistamine, an HIV antiretroviral drug, or a protease inhibitor.

4. The method of claim 1 wherein R11, R12, and R13 are independently methyl, ethyl, propyl, allyl, ether, sulfhydryl, amino, or hydroxyl groups; L is —(CH₂)₂— or —(CH₂)₃—; and R₁ and R₃₋₈ are hydrogen or methyl.

5. The method of claim 1 wherein L-N⁺(R11, R12, R13) is choline.

6. The method of claim 1 wherein the compound is a thioxanthone.

7. The method of claim 6 wherein R9 and R10 considered together are ═O and R2 is —X-L-N⁺(R11, R12, R13)Z-.

8. The method of claim 6 wherein the compound is [3-(3,4-dimethyl-9-oxo-9H-thioxanthen-2-yloxy)-2-hydroxypropyl] trimethylammonium chloride.

9. The method of claim 6 wherein the compound is N,N,-diethyl-N-methyl-2-[9-oxo-9H-thioxanthen-2-yl)methoxy]ethanaminium iodide.

10. The method of claim 1 wherein the compound is a thioxanthene.

11. The method of claim 10 wherein R2 is 0 or X and R9 and R10 considered together are ═CH-L-N⁺(R11, R12, R13); L is —(CH₂)₂— or —(CH₂)₃—; and R1 and R3-8 are hydrogen or methyl.

12. The method of claim 10 wherein the compound is N,N,N-trimethyl-3-(9H-thioxanthen-9-ylidene)-propane-1-aminium iodide.

13. The method of claim 10 wherein the compound is N,N-Diethyl-N-allyl-3-(2-methyl-9H-thioxanthen-9-ylidene)-propane-1-aminium bromide.

14. The method of claim 1 wherein the mammal is on a calorie restricted or other type of weight loss-reducing diet.

15. The method of claim 1 wherein the heterocyclic compound is administered to the mammal prior to and/or after performing bariatric surgery.

16. The method of claim 1 wherein the heterocyclic compound is administered to the mammal together with the appetite suppressant Phentermine or another anti-obesity drug selected from Orlistat, Sibutramine, or a CB1 receptor-interacting drug.

17. The method of claim 1 wherein the heterocyclic compound is administered together with an alkaline phosphatase.

18. The method of claim 1 wherein the heterocyclic compound is administered orally in the form of a tablet, gel capsule, or liquid, or in any other suitable form at a dose between 100-mg to 2,000-mg per m² body surface of the mammal once, twice, or thrice daily, or three-times a week, or intermittently.

19. The method of claim 1 wherein the heterocyclic compound is dissolved in a suitable physiologically compatible liquid carrier, such as physiological saline, and administered via an injection method selected from intravenous, intraarterial, subcutaneous, intraperitoneal, intradermal, or intramuscular, or via infusion, or by using a subcutaneously inserted osmotic minipump to ensure controlled release.

20. The method of claim 19 wherein the heterocyclic compound is administered at a dose between 50-mg to 1,000-mg per m² body surface of the mammal once, twice, or thrice daily, or three-times a week, or intermittently.

21. The method of claim 19 wherein the alkaline phosphatase is administered once, twice, or three-times a week using a dose range from 100 mg to 2,000 mg per m² body surface while the heterocyclic compound is administered daily or intermittently orally or by an injection method at a dose of 50-mg to 2,000-mg per m² body surface of the mammal.

22. The method of claim 1, wherein when one of V or Y is —N, -L-N⁺(R11, R12, R13) is linked to the —N.

23. The method of claim 1, wherein when both of V or Y are —N, -L-N(R11, R12, R13) is linked to both V and Y.
